(19) United States
(12) Patent Application Publication
(10) Pub. No.: US 2026/0061406 A1
(43) Pub. Date: Mar. 5, 2026

(54) IRON CONTAINING SSZ-42 MOLECULAR SIEVE CATALYST FOR HYDROPROCESSING

(71) Applicant: Chevron U.S.A. Inc., San Ramon, CA (US)

(72) Inventors: Stacey Ian ZONES, San Fransciso, CA (US); Cong-Yan CHEN, Kensington, CA (US)

(73) Assignee: Chevron U.S.A. Inc., San Ramon, CA (US)

(21) Appl. No.: 19/314,584

(22) Filed: Aug. 29, 2025

Related U.S. Application Data
(60) Provisional application No. 63/689,159, filed on Aug. 30, 2024.

(51) Int. Cl. B01J 29/76; B01J 37/02; B01J 37/08; B01J 37/30; C01B 39/48; C07C 5/27

(52) U.S. Cl. CPC B01J 29/76; B01J 37/0201; B01J 37/08; B01J 37/30; C01B 39/48; C07C 5/2775; C01P 2002/72; C07C 2529/76

(57) ABSTRACT

Provided is a new crystalline zeolite Fe SSZ-42 prepared by processes for preparing crystalline molecular sieves, particularly large pore zeolites, using an organic templating agent selected from the group consisting of N-benzyl-1,4-diazabicyclo[2.2.2]octane cations and N-benzyl-1-azabicyclo[2.2.2]octane cations.

### CROSS REFERENCE TO RELATED APPLICATIONS

This application claims priority to U.S. Provisional Application Ser. No. 63/689,159 filed Aug. 30, 2024, the complete disclosure of which is incorporated herein by reference in its entirety.

### FIELD OF THE INVENTION

An iron SSZ-42 zeolite catalyst useful in hydroconversion processes, especially hydroisomerization.

### BACKGROUND

In conventional usage the term “molecular sieve” refers to a material having a fixed, open-network structure, usually crystalline, that may be used to separate hydrocarbons or other mixtures by selective occlusion of one or more of the constituents, or may be used as a catalyst in a catalytic conversion process. The term “zeolite” refers to a molecular sieve containing a silicate lattice, usually in association with some aluminum, boron, gallium, iron, and/or titanium. In the following discussion and throughout this disclosure, the terms molecular sieve and zeolite will be used more or less interchangeably. One skilled in the art will recognize that the teachings relating to zeolites are also applicable to the more general class of materials called molecular sieves.

Natural and synthetic crystalline molecular sieves are useful as catalysts and adsorbents. Each crystalline molecular sieve is distinguished by a crystal structure with an ordered pore structure, and is characterized by a unique X-ray diffraction pattern. Thus, the crystal structure defines cavities and pores which are characteristic of the different species. The adsorptive and catalytic properties of each crystalline molecular sieve are determined in part by the dimensions of its pores and cavities. Accordingly, the utility of a particular molecular sieve in a particular application depends at least partly on its crystal structure.

Because of their unique sieving characteristics, as well as their catalytic properties, crystalline molecular sieves are especially useful in applications such as hydrocarbon conversion, gas drying and separation. Although many different crystalline molecular sieves have been disclosed, there is a continuing need for new zeolites with desirable properties for gas separation and drying, hydrocarbon and chemical conversions, and other applications.

Crystalline aluminosilicates are usually prepared from aqueous reaction mixtures containing alkali or alkaline earth metal oxides, silica, and alumina. Crystalline borosilicates are usually prepared under similar reaction conditions except that boron is used in place of aluminum. By varying the synthesis conditions and the composition of the reaction mixture, different zeolites can often be formed.

Organic templating agents are believed to play an important role in the process of molecular sieve crystallization. Organic amines and quaternary ammonium cations were first used in the synthesis of zeolites in the early 1960s as reported by R. M. Barrer and P. J. Denny in J. Chem. Soc. 1961 at pages 971-982. This approach led to a significant increase in the number of new zeolitic structures discovered as well as an expansion in the boundaries of composition of the resultant crystalline products.

Previously, products with low silica to alumina ratios (SiO₂/Al₂O₃≤10) had been obtained, but upon using the organocations as components in the starting gels, zeolites with increasingly high SiO₂/Al₂O₃ were realized. Some of these materials are summarized by R. M. Barrer 1982, Hydrothermal Chemistry of Zeolites, New York: Academic Press, Inc.

Unfortunately, the relationship between structure of the organocation and the resultant zeolite is far from predictable, as evidenced by the multitude of products which can be obtained using a single quaternary ammonium salt as reported by S. I. Zones et al., 1989, Zeolites: Facts, Figures, Future, ed. P. A. Jacobs and R. A. van Santen, pp. 299-309, Amsterdam: Elsevier Science Publishers, or the multitude of organocations which can produce a single zeolitic product as reported by R. M. Barrer, 1989, Zeolite Synthesis, ACS Symposium 398, ed. M. L. Occelli and H. E. Robson, pp. 11-27, American Chemical Society.

Thus, it is known that organocations exert influence on the zeolite crystallization process in many unpredictable ways. Aside from acting in a templating role, the organic cation's presence also greatly affects the characteristics of the gel. These effects can range from modifying the gel pH to altering the interactions of the various components via changes in hydration (and thus solubilities of reagents) and other physical properties of the gel. Accordingly, investigators have now begun to consider how the presence of a particular quaternary ammonium salt influences many of these gel characteristics in order to determine more rigorously how such salts exert their templating effects.

U.S. Pat. No. 5,194,235, issued Mar. 6, 1993 to Zones, discloses the use of a templating agent known as DABCO-Cn-diquat to prepare the zeolite SSZ-16. This templating agent has the following formula:

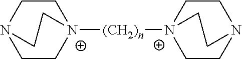

wherein n is 3, 4 or 5.

In summary, a variety of templates have been used to synthesize a variety of molecular sieves, including zeolites of the silicate, aluminosilicate, and borosilicate families. However, the specific zeolite which may be obtained by using a given template is at present unpredictable. In fact, the likelihood of any given organocation serving as an effective template useful in the preparation of a molecular sieve is conjectural at best. In particular, organocation templating agents have been used to prepare many different combinations of oxides with molecular sieve properties, with silicates, aluminosilicates, aluminophosphates, borosilicates and silicoaluminophosphates being well known examples.

U.S. Pat. No. 5,653,956 describes a SSZ-42 zeolite. A Fe SSZ-42 and how to make it, however, is not described. Neither are the benefits of using an iron SSZ-42 zeolite in a hydroconversion reaction as a catalyst.

Ongoing efforts to improve base stock yield of dewaxing processes have included upgrading hydrocarbon feedstocks and enhancement of activity of the catalyst used in the dewaxing process. Dewaxing by isomerization includes a catalytic reaction dependent upon catalyst pore structure and framework composition as well as the size of reactant and product molecules. Zeolite-based catalysts having a noble metal as a hydrogenation component have been shown to improve hydrocarbon conversion performance and isomerization of n-paraffins. Improvements in such catalysts would be welcome by the industry.

### SUMMARY

Provided is a Fe SSZ-42 zeolite having the X-ray diffraction lines of Table I. Upon calcining, the zeolite exhibits the X-ray diffraction lines of Table II.

There is further provided in one embodiment a zeolite having a composition, as-synthesized and in the anhydrous state, in terms of mole ratios as follows:

| SiO₂/Fe₂O₃ | Greater than or equal to 15 |
|---|---|
| SiO₂/MₓO | Greater than or equal to 45 |
| SiO₂/Q | 10-40 |

wherein Q is comprised of cations selected from the group consisting of N-benzyl-1,4-diazabicyclo[2.2.2]octane cations and N-benzyl-1-azabicyclo[2.2.2]octane cations, M is an alkali metal cation or alkaline earth metal cation, X is 1 or 2, and with the zeolite having the X-ray diffraction lines of Table I.

In another embodiment, there is also provided a zeolite prepared by thermally treating (calcining) an iron SSZ-42 zeolite having the X-ray diffraction lines of Table I at a temperature of from about 200° C. (392° F.) to about 800° C. (1472° F.), the thus-treated zeolite having the X-ray diffraction lines of Table II. One embodiment includes the hydrogen form of this thus-prepared zeolite, which hydrogen form is prepared by ion exchanging with an acid or with a solution of an ammonium salt followed by a second thermal treatment.

### BRIEF DESCRIPTION OF THE DRAWINGS

FIG. 1 graphically depicts the conversion yield over item for n-decane conversion of Pd Fe SSZ-42.

FIG. 2 graphically depicts the distribution vs. conversion of the n-decane.

FIG. 3 graphically depicts the type of conversion yield vs. conversion.

FIG. 4 graphical depicts the conversion related to temperature ° F.

FIG. 5 graphical depicts the conversion related to temperature ° C.

FIG. 6 shows the acidity measurement for Fe SSZ-42, greater than 200

micromoles recorded for n-propylamine.

### DETAILED DESCRIPTION

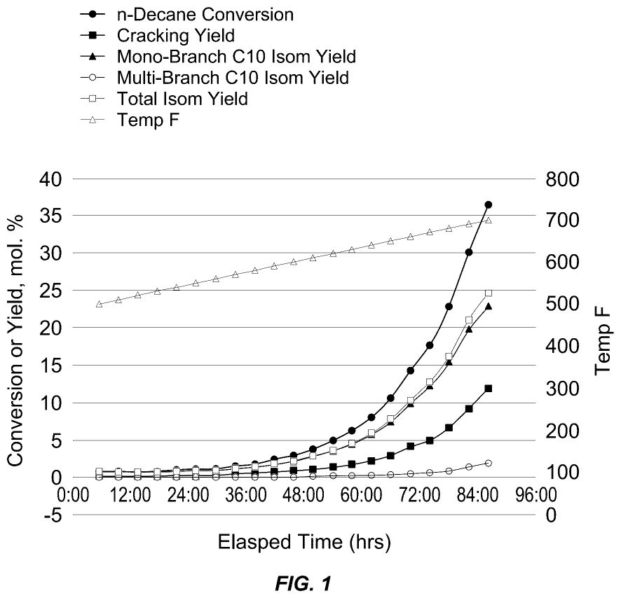

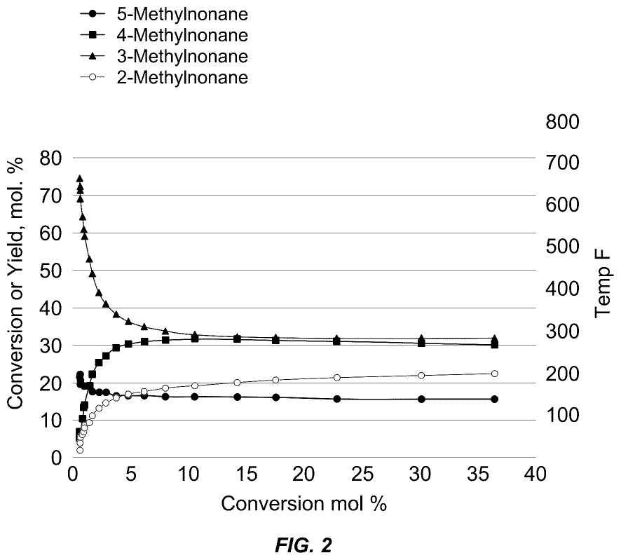

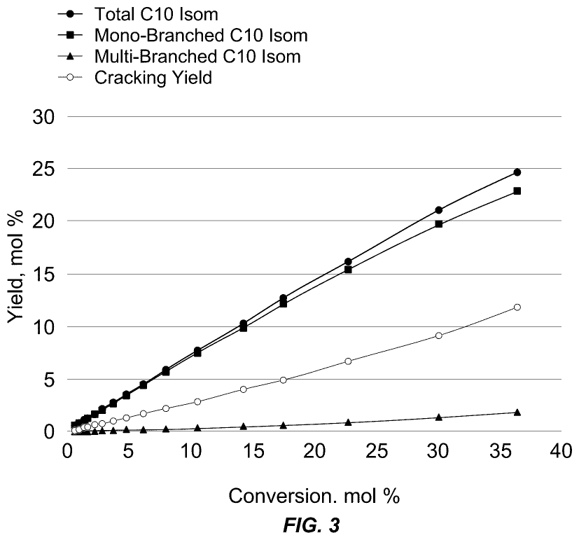

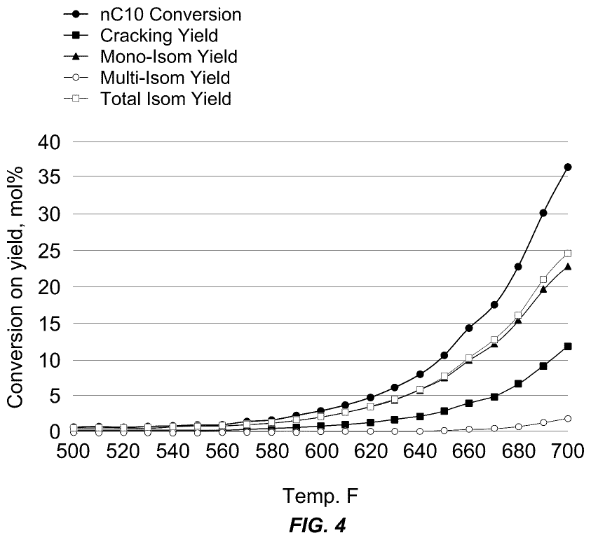

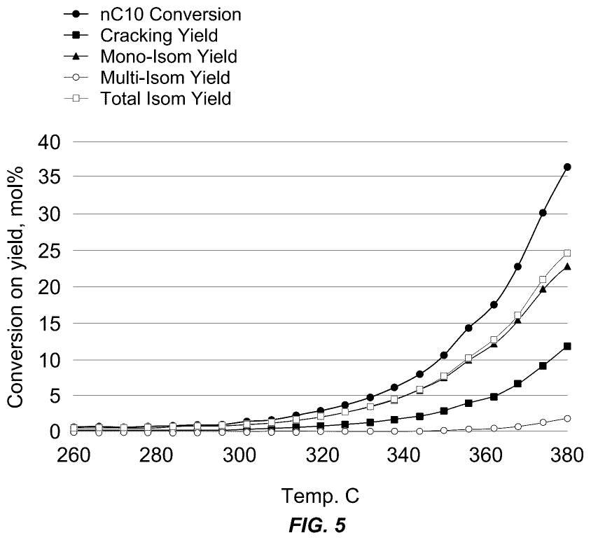

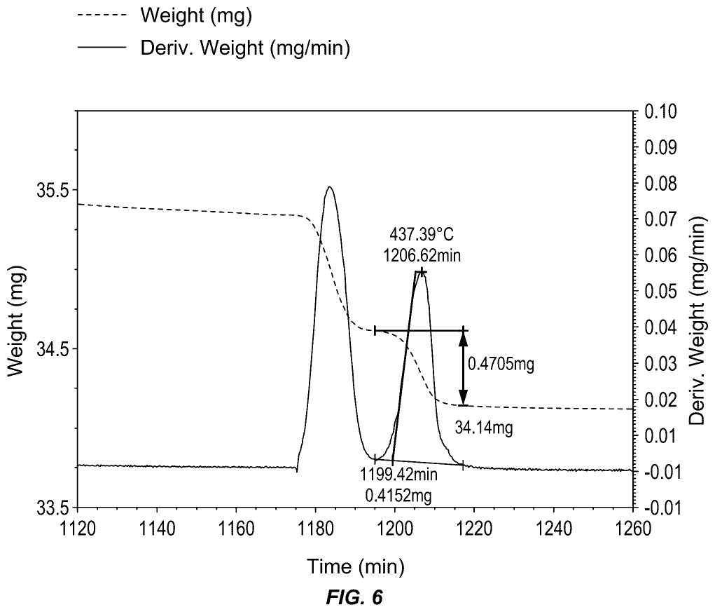

The present Fe SSZ-42 is a zeolite with a one dimensional 12-ring pore, but it is periodically crossed by what amounts to side pockets: not true continuous pores. However, one can imagine that the side pocket areas entering into the main channel can enlarge the space for transition states for catalytic reactions. Data relating to n-decane (nC₁₀) isomerization is shown in the sequence FIGS. 1-5. There are a few features which are remarkable. First at about 30% conversion the isomerization/cracking ratio is about 2.5, giving mainly isomerized nonanes. Second there are almost no di-branched products while there is sufficient space to create them internally. One speculation is that di-branched, as they form are then easily hydrocracked to smaller fragments. Interestingly the acidity for the zeolite is a good population. The data is shown in FIG. 6, and the quantitative assessment has the sites slightly above 200 micromoles.

The iron SSZ-42 zeolites can be prepared from an aqueous solution comprising sources of silicon oxide, an alkali or alkaline earth metal oxide, the templating agent, and a source of iron, generally an iron salt. The reaction mixture should have a composition, in terms of mole ratios, within the ranges shown in Table A.

TABLE A
SSZ-42 REACTION MIXTURE

| | Broad | Preferred |
|---|---|---|
| SiO₂/Fe₂O₃ | 5 and greater | 15 and greater |
| | (to about 100) | (to about 100) |
| OH−/SiO₂ | 0.05 to 0.50 | 0.5 to 0.30 |
| Q/SiO₂ | 0.10 to 1.0 | 0.10 to 0.25 |
| M+/SiO₂ | 0.01 to 0.50 | 0.03 to 0.10 |
| H₂O/SiO₂ | 15 to 100 | 20 to 50 |
| Q/Q + M⁺ | 0.50 to 0.95 | 0.66 to 0.90 |

wherein Q is comprised of cations selected from the group consisting of N-benzyl-1,4-diazabicyclo[2.2.2]octane cations and N-benzyl-1-azabicyclo[2.2.2]octane cations, and M is an alkali metal cation or alkaline earth metal cation.

The iron SSZ-42 can be made essentially aluminum free, i.e., having a silica to alumina mole ratio of x. The term “essentially alumina-free” is used because it is difficult to prepare completely aluminum-free reaction mixtures for synthesizing these materials. Especially when commercial silica sources are used, aluminum is almost always present to a greater or lesser degree. The hydrothermal reaction mixtures from which the essentially alumina-free crystalline siliceous molecular sieves may be prepared can be referred to as being substantially alumina free. By this usage is meant that no aluminum is intentionally added to the reaction mixture, e.g., as an alumina or aluminate reagent, and that to the extent aluminum is present, it occurs only as a contaminant in the reagents. An additional method of increasing the mole ratio of silica to alumina is by using standard acid leaching or chelating treatments. However, essentially aluminum-free SSZ-42 can be synthesized directly using essentially aluminum-free silicon sources as the only tetrahedral metal oxide component.

It is believed that the iron SSZ-42 is comprised of a framework structure or topology which is characterized by its X-ray diffraction pattern. The iron SSZ-42 zeolites, as-synthesized, have a crystalline structure whose X-ray powder diffraction pattern exhibit the characteristic lines shown in Table I, and includes iron.

TABLE I
AS SYNTHESIZED IRON SSZ-42

| 2 Theta | d/n | 100I/Iₒ |
|---|---|---|
| 8.26 | 10.70 | 70 |
| 9.76 | 9.05 | 7 |
| 16.54 | 5.355 | 15 |
| 19.16 | 4.628 | 21 |
| 20.64 | 4.300 | 100 |
| 21.58 | 4.115 | 23 |
| 21.80 | 4.074 | 49 |
| 23.72 | 3.748 | 10 |
| 23.92 | 3.717 | 35 |
| 24.96 | 3.565 | 11 |
| 25.38 | 3.506 | 12 |
| 26.24 | 3.393 | 26 |
| 26.78 | 3.326 | 26 |
| 29.46 | 3.030 | 18 |

The variation in the scattering angle (two theta) measurements, due to instrument error and to differences between individual samples, is estimated at ±0.20 degrees.

The X-ray diffraction pattern of Table I is representative of as-synthesized iron SSZ-42 zeolites. Minor variations in the diffraction pattern can result from variations in the silica-to-alumina or silica-to-boron mole ratio of the particular sample due to changes in lattice constants. In addition, sufficiently small crystals will affect the shape and intensity of peaks, leading to significant peak broadening.

After calcination, the iron SSZ-42 zeolites have a crystalline structure whose X-ray powder diffraction pattern include the characteristic lines shown in Table II:

TABLE II
CALCINED SSZ-42

| 2 Theta | d/n | 100I/Iₒ |
|---|---|---|
| 8.22 | 10.75 | 100 |
| 9.76 | 9.06 | 13 |
| 16.42 | 5.394 | 3 |
| 19.22 | 4.615 | 7 |
| 20.48 | 4.333 | 30 |
| 20.84 | 4.259 | 25 |
| 21.48 | 4.134 | 7 |
| 21.72 | 4.088 | 16 |
| 23.68 | 3.754 | 6 |
| 24.06 | 3.696 | 15 |
| 24.94 | 3.568 | 10 |
| 25.40 | 3.504 | 6 |
| 26.60 | 3.348 | 20 |
| 29.56 | 3.019 | 10 |

The variation in the scattering angle (two theta) measurements, due to instrument error and to indifferences between individual samples, is estimated at ±0.20 degrees.

Representative peaks from the X-ray diffraction pattern of calcined iron SSZ-42 are shown in Table II. Calcination can also result in changes in the intensities of the peaks as compared to patterns of the “as-synthesized” material, as well as minor shifts in the diffraction pattern. The zeolite produced by exchanging the metal or other cations present in the zeolite with various other cations (such as H⁺ or NH₄⁺) yields essentially the same diffraction pattern, although again, there may be minor shifts in the interplanar spacing and variations in the relative intensities of the peaks. Notwithstanding these minor perturbations, the basic crystal lattice remains unchanged by these treatments.

The X-ray powder diffraction patterns were determined by standard techniques. The radiation was the K-alpha/doublet of copper. A scintillation counter spectrometer with a strip-chart pen recorder was used. The peak heights I and the positions, as a function of 2θ where θ is the Bragg angle, were read from the relative intensities, I/Io where Io is the intensity of the strongest line or peak, and d, the interplanar spacing in Angstroms corresponding to the recorded lines, can be calculated.

In preparing the iron SSZ-42 zeolites, an N-benzyl-1,4-diazabicyclo[2.2.2]octane cation or N-benzyl-1-azabicyclo[2.2.2]octane cation may be used as a crystallization template in a manner known in the molecular sieve art. Thus, in general, SSZ-42 is prepared by contacting an active source of one or more oxides selected from the group consisting of monovalent element oxides, divalent element oxides, trivalent element oxides, and tetravalent element oxides with an organocation templating agent and an iron salt. Any iron salt can be used. Examples include ferric nitrate, ferric sulfate, ferric sulfite, ferric chloride, ferric acetate, and an iron EDTA complex. But suitable iron salts are known.

In practice, the iron SSZ-42 is prepared by a process comprising:
(a) preparing an aqueous solution containing sources of the oxides noted above and at least one N-benzyl-1,4-diazabicyclo[2.2.2]octane cation or N-benzyl-1-azabicyclo[2.2.2]octane cation templating agent having an anionic counterion which is not detrimental to the formation of SSZ-42, and an iron salt:
(b) maintaining the aqueous solution under conditions sufficient to form crystals of iron SSZ-42; and
(c) recovering the crystals of iron SSZ-42.

The N-benzyl-1,4-diazabicyclo[2.2.2]octane cation and N-benzyl-1-azabicyclo[2.2.2]octane cation templating agents which have been found to produce the iron SSZ-42 have the following general formulas:

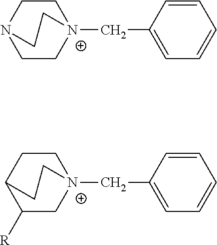

where R is —H, —OH or —NH₂.

Examples of the N-benzyl-1,4-diazabicyclo[2.2.2]octane cation templating agents useful in this invention include, but are not limited to, N-benzyl-1,4-diazabicyclo[2.2.2]octane cation, and examples of the N-benzyl-1-azabicyclo[2.2.2]octane cation templating agents useful in this invention include, but are not limited to, N-benzyl-1-azabicyclo[2.2.2]octane cation and N-benzyl-3-hydroxy-1-azabicyclo[2.2.2]octane cation.

Typical sources of silicon oxide include silicates, silica hydrogel, silicic acid. fumed silica, colloidal silica, tetra-alkyl orthosilicates, and silica hydroxides.

Typically, an alkali metal hydroxide and/or an alkaline earth metal hydroxide, such as the hydroxide of sodium, potassium, lithium, cesium, rubidium, calcium, and magnesium, is used in the reaction mixture: however, this component can be omitted so long as the equivalent basicity is maintained. The templating agent may be used to provide hydroxide ion. Thus, it may be beneficial to ion exchange, for example, a hydroxide anion for a halide ion in the templating agent, thereby reducing or eliminating the alkali or alkaline earth metal hydroxide quantity required. The alkali metal cation or alkaline earth cation may be part of the as-synthesized crystalline oxide material, in order to balance valence electron charges therein.

The reaction mixture is maintained at an elevated temperature until the crystals of the SSZ-42 zeolite are formed. This hydrothermal crystallization is usually conducted under autogenous pressure, at a temperature between 100° C. (212° F.) and 200° C. (392° F.), preferably between 135° (275° F.) and 180° C. (356° F.). The crystallization period is typically greater than 1 day and preferably from about 3 days to about 7 days. The zeolite can be prepared with or without mild stirring or agitation.

During the hydrothermal crystallization step, the iron SSZ-42 crystals can be allowed to nucleate spontaneously from the reaction mixture. However, the use of SSZ-42 crystals as seed material can be advantageous in decreasing the time necessary for complete crystallization to occur. In addition, seeding can lead to an increased purity of the product obtained by promoting the nucleation and/or formation of SSZ-42 over any undesired phases. When used as seeds, SSZ-42 crystals are added in an amount between 0.1 and 10% of the weight of silica used in the reaction mixture.

Once the zeolite crystals have formed, the solid product is separated from the reaction mixture by standard mechanical separation techniques such as filtration. The crystals are water-washed and then dried, e.g., at 90° C. (194° F.) to 150° C. (302° F.) for from 8 to 24 hours, to obtain the as-synthesized, iron SSZ-42 zeolite crystals. The drying step can be performed at atmospheric pressure or under vacuum.

Crystalline iron SSZ-42 can be used as-synthesized, or preferably can be thermally treated (calcined). Usually, it is desirable to remove the alkali or alkaline earth metal cation by ion exchange and replace it with hydrogen, ammonium, or any desired metal ion. The zeolite can be used in intimate combination with hydrogenating components, such as tungsten, vanadium, molybdenum, rhenium, nickel, cobalt, chromium, manganese, or a noble metal, such as palladium or platinum, for those applications in which a hydrogenation-dehydrogenation function is desired.

Metals may also be introduced into the zeolite by replacing some of the cations in the zeolite with metal cations via ion exchange techniques. Typical replacing cations can include metal cations, e.g., rare earth, Group IIA and Group VIII metals, as well as their mixtures. Of the replacing metallic cations, cations of metals such as rare earth, Mn, Ca, Mg, Zn, Cd, Pt, Pd, Ni, Co, Ti, Al, Sn, and Fe are particularly preferred.

The hydrogen, ammonium, and metal components can be ion-exchanged into the SSZ-42. The zeolite can also be impregnated with the metals, or the metals can be physically and intimately admixed with the zeolite using standard methods known to the art.

The iron SSZ-42 zeolites have been found useful in hydrocarbon conversion reactions. Hydrocarbon conversion reactions are chemical and catalytic processes in which carbon containing compounds are changed to different carbon containing compounds. Examples of hydrocarbon conversion reactions in which the iron SSZ-42 are expected to be useful include catalytic cracking, hydrocracking, dewaxing, alkylation, and olefin and aromatics formation reactions. The catalysts are also expected to be useful in other petroleum refining and hydrocarbon conversion reactions such as isomerizing n-paraffins and naphthenes, polymerizing and oligomerizing olefinic or acetylenic compounds such as isobutylene and 1-butene, reforming, alkylating, isomerizing polyalkyl substituted aromatics (e.g., m-xylene), and disproportionating aromatics (e.g., toluene) to provide mixtures of benzene, xylenes and higher methylbenzenes and oxidation reactions. The iron SSZ-42 catalysts have high selectivity, and under hydrocarbon conversion conditions can provide a high percentage of desired products relative to total products. In one embodiment, the iron SSZ-42 catalyst is most useful in hydroisomerization.

SSZ-42 zeolites can be used in processing hydrocarbonaceous feedstocks. Hydrocarbonaceous feedstocks contain carbon compounds and can be from many different sources, such as virgin petroleum fractions, recycle petroleum fractions, shale oil, liquefied coal, tar sand oil, and, in general, can be any carbon containing fluid susceptible to zeolitic catalytic reactions. Depending on the type of processing the hydrocarbonaceous feed is to undergo, the feed can contain metal or be free of metals, it can also have high or low nitrogen or sulfur impurities. It can be appreciated, however, that in general processing will be more efficient (and the catalyst more active) the lower the metal, nitrogen, and sulfur content of the feedstock.

The conversion of hydrocarbonaceous feeds can take place in any convenient mode, for example, in fluidized bed, moving bed, or fixed bed reactors depending on the types of process desired. The formulation of the catalyst particles will vary depending on the conversion process and method of operation.

The reactions can be performed using the present catalyst containing a metal, e.g., a Group VIII metal such as platinum or palladium. Such catalyst are especially useful for hydroisomerization.

The iron SSZ-42 can be used in hydrocarbon conversion reactions with active or inactive supports, with organic or inorganic binders, and with and without added metals. These reactions are well known to the art, as are the reaction conditions.

### EXAMPLE 1

### Synthesis of Fe-SSZ-42

To a 23-mL Teflon liner were added 4.14 mmole of N-benzyl-1,4-diazabicyclo[2.2.2]octane hydroxide (in 4.4 g of solution), 2 mmole of aqueous 1 M NaOH solution, 2 g of deionized water and 0.10 g of ferric nitrate nonahydrate. Thereafter, 0.90 g of CAB-O-SILR fumed silica (Cabot Corp.) were added. The liner was then capped and sealed within a 23 mL Parr autoclave vessel. The autoclave vessel was then heated in a Blue M convection oven under tumbling conditions (43 rpm) for 7 days at 160° C. The product was isolated by filtration, washed with deionized water, and then dried in an oven at 95° C.

Powder XRD (not shown) indicated that the as-synthesized product was SSZ-42.

Elemental analysis by Inductively Coupled Plasma—Atomic Emission Spectroscopy (ICP-AES) of the as-synthesized product indicates 1.60% Fe and 43.2% Si.

A portion of the as-synthesized product was activated by calcining in air. The solid was heated in a muffle furnace to 540° C. at a rate of 1° C./min and held at 540° C. for 5 hours. Once the solid had cooled to room temperature, it was ion-exchanged to the NH₄-form by heating in a solution of ammonium nitrate (typically, 1 g NH₄NO₃/1 g zeolite in 10 ml deionized water at 95° C. for at least 2 hours). The zeolite was then filtered. This was repeated twice for a total of 3 exchanges. The zeolite was washed with deionized water to a conductivity of less than 50 μS/cm and dried in air at 95° C. The resulting NH₄-form zeolite was converted to the H-form by calcining, in air, as described above.

Analysis by n-propy lamine temperature-programmed desorption showed that the activated H-form zeolite had an acid site density of greater than 200 μmol H⁺/g. This is shown in FIG. 6.

### EXAMPLE 2

### Hydroconversion of n-Decane

Calcined Fe-SSZ-42 product (NH₄-form) was impregnated with palladium at a loading of 0.5 wt. % using the required amount of tetraaminepalladium (II) nitrate dissolved in deionized water. The impregnated sample was washed to a conductivity of less than 50 μS/cm, dried and calcined in air at 482° C. for 3 hours. The resulting powdered catalyst material was pelletized at 5 kpsi, crushed and sieved to 20-40 mesh.

0.5 g of catalyst was charged into the center of a 23 inch-long×¼ inch outside diameter stainless steel reactor tube with alundum loaded upstream of the catalyst for preheating the feed. The run conditions were as follows: a total pressure of 1200 psig, a down-flow hydrogen rate of 8.3 mL/minute (when measured at 1 atmospheric pressure and 25° C.) and a down-flow n-decane liquid feed rate of 0.66 cm³/h. The catalyst was first reduced in flowing hydrogen at about 315° C. for 1 h and then tested at various reaction temperatures. Products were analyzed by on-line capillary gas chromatography (GC) once every one hour. Raw data from the GC was collected by an automated data collection/processing system and hydrocarbon conversions were calculated from the raw data.

Conversion is defined as the amount of n-decane reacted to produce other products (including iso-C₁₀). Yields are expressed as mol % of products other than n-decane and include iso-C₁₀ isomers as a yield product. The data for the n-decane isomerization is graphically shown in FIGS. 1-5. The hydroisomerization provided excellent results.

As used in this disclosure the word “comprises” or “comprising” is intended as an open-ended transition meaning the inclusion of the named elements, but not necessarily excluding other unnamed elements. The phrase “consists essentially of’ or “consisting essentially of’ is intended to mean the exclusion of other elements of any essential significance to the composition. The phrase “consisting of’ or “consists of’ is intended as a transition meaning the exclusion of all but the recited elements with the exception of only minor traces of impurities.

All patents and publications referenced herein are hereby incorporated by reference to the extent not inconsistent herewith. It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present invention, but they are not essential to its practice. It is therefore to be understood that the invention may be practiced otherwise that as specifically described without actually departing from the spirit and scope of the present invention as defined by the appended claims.

What is claimed is:

1. A zeolite having a composition, as-synthesized and in the anhydrous state, in terms of mole ratios as follows:

| SiO₂/Fe₂O₃ | Greater than or equal to 15 |
|---|---|
| SiO₂/MₓO | Greater than or equal to 45 |
| SiO₂/Q | 10-40 |

wherein Q is comprised of cations selected from the group consisting of N-benzyl-1-azabicyclo[2.2,2]octane cations having the structure 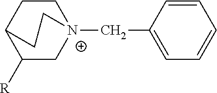
where R is —OH or —NH₂, and N-benzyl-1,4-diazabicyclo[2.2.2]octane cations, M is an alkali metal cation or alkaline earth metal cation, x is 1 or 2, and said zeolite having the X-ray diffraction lines of Table I, while containing iron.

2. A zeolite according to claim 1 wherein the oxides comprise silicon oxide and iron oxide.

3. A zeolite according to claim 2, wherein said zeolite is predominantly in the hydrogen form.

4. A zeolite according to claim 1, wherein said zeolite is predominantly in the hydrogen form.

5. The zeolite of claim 1 wherein the crystalline material has, after calcination, the X-ray diffraction lines of Table II.

6. A process of hydroconversion comprising reacting hydrocarbons in the presence of a catalyst comprising the zeolite of claim 1.

7. The process of claim 6, wherein the zeolite has been calcined.

8. The process of claim 6, wherein the zeolite is impregnated with platinum or palladium, or a mixture thereof.

9. The process of claim 8, wherein the zeolite is impregnated with palladium.

10. The process of claim 6, wherein the process comprises hydroisomerization.

11. The process of claim 9, wherein the process comprises hydroisomerization.
